(19) United States
(12) Patent Application Publication
(10) Pub. No.: US 2014/0277328 A1
(43) Pub. Date: Sep. 18, 2014

(54) COMPOSITE MATERIAL AND USES THEREOF

(71) Applicant: ST. JUDE MEDICAL SYSTEMS AB, Uppsala (SE)

(72) Inventors: Andreas ÖRNBERG, West Saint Paul, MN (US); Anna Norlin-Weissenrieder, Lidingo (SE); Jonas Weissenrieder, Lidingo (SE)

(73) Assignee: ST. JUDE MEDICAL SYSTEMS AB, Uppsala (SE)

(21) Appl. No.: 13/829,033

(22) Filed: Mar. 14, 2013

(51) Int. Cl. A61L 27/42; A61L 31/12

(52) U.S. Cl. CPC A61L 27/427; A61L 31/124
USPC 623/1.1; 75/249; 75/232; 419/1; 419/26; 419/28; 606/213; 606/151

(57) ABSTRACT

A composite material for a medical implant includes a matrix of magnesium or magnesium alloy, and a catalyst which is dispersed within the matrix. The catalyst has the capacity to reduce an amount of hydrogen gas released from the matrix when the matrix is being degraded inside the patient.

### TECHNICAL FIELD

The present disclosure relates generally to a magnesium based composite material comprising a catalyst, and further to biologically degradable medical implants comprising said composite material.

### BACKGROUND

Temporary implants that are biologically degradable offer the advantage that they need not be removed in a second surgical intervention after serving their purpose, which makes them both convenient for the patient and more economical. Biodegradable polymers are commonly used for implantation purposes. This group of polymers often exhibit desirable properties in terms of degradation rate and biocompatibility, but they often lack the necessary mechanical stability, and it is known that metal-based biodegradable implants may offer more suitable mechanical properties than polymers. For example, magnesium (Mg) and magnesium based alloys have become interesting candidates in the field of biodegradable implant materials.

In general, magnesium and its alloys have great potential as biodegradable materials, since magnesium is an essential element in the human body. Mg degrades in aqueous solutions and offers a mechanically more stable construction of the implant than degradable polymeric materials (Hanzi et al., Acta Biomaterialia, 2009, 5:162-171). The entire contents of this publication is incorporated herein by reference for the Mg and Mg based materials and related processes described therein. Mg and its alloys have been used for biodegradable orthopaedic implants, and their application to coronary stents and other vascular devices is currently being investigated. It has been considered that the mechanical and corrosion properties of pure magnesium do not suit the requirements for stent material. However, magnesium alloys are seen as promising biodegradable implant materials (Mani et al., Biomaterials, 2007, 28: 1689-1710). The entire contents of this publication is incorporated herein by reference for the Mg and Mg based materials and related processes described therein.

Magnesium and its alloys exhibit one major drawback when allowed to degrade in the body by its natural reaction. Degradation of magnesium in aqueous solutions at pH values below 11.5 (typically the pH within a body is less than 11.5) is associated with the formation of hydroxide ions and gaseous hydrogen, as described by the following electrochemical reaction:

Mg+2H₂O→Mg²⁺+2OH⁻+H₂

This mechanism is highly insensitive to the oxygen concentration. In vivo, hydrogen ions can cause harm to tissues as the pH value increases locally, and hydrogen gas formation can cause gas pockets to form around the implant and thereby cause damage to the surrounding tissue or create gas bubble formation in the blood, which is dangerous to the patient.

The conventional method of solving the problems associated with hydrogen release from magnesium implants consists of slowing down the degradation rate and hence reducing the release rate of hydrogen gas and hydroxide ions. This is commonly done by enhancing the corrosion resistance of the material by various methods, such as alloying, modifying their microstructure or their surface, including the application of conversion coatings (Staiger et al., Biomaterials, 2006, 27: 1728-1734; Guangling Song, Corrosion Science, 2007, 49: 1696-1701; Wang et al., Advanced Materials Research, 2008, 32: 207-210).

Coating the metallic surface with other materials to alter its surface characteristics without interfering with the bulk properties of the metal implant has been one rational approach to address the issue relating to the compatibility of metallic stents with tissue and blood. The coating can improve the surface properties significantly; surface energy can be reduced, surface texture can be smoothened, surface potential can be neutralised and the stability of the surface oxide layer can be enhanced. There are many inorganic coating materials which are potentially suitable for the treatment of medical implant surfaces. For example, gold and iridium oxide have been used as stent inorganic coating materials (Mani et al., Biomaterials, 2007, 28: 1689-1710), however not on magnesium-based implants.

None of the methods mentioned above reduce the total amount of hydrogen released during the degradation of the magnesium present in the implant. These methods only slow the release rate down as a result of the slower degradation rate.

Another method for addressing hydrogen evolution is the development of magnesium based metallic glasses. This material has extended solubility for alloying elements and a homogeneous single-phase structure which can lead to altered corrosion behavior and reduction of hydrogen evolution (Bruno Zberg, et al., Nature Materials, 2009, 8: 887-891). Often metallic glasses are difficult and expensive to manufacture in larger quantities as the material has to be cooled at a very rapid rate to obtain the amorphous structure. It makes it especially challenging to manufacture thick materials as the thicker it is, the more difficult it is to maintain an appropriate cooling rate within the bulk of the material. Another disadvantage with metallic glass is that it can be difficult to maintain its amorphous structure when it is subjected to processing steps, like laser cutting, annealing and shaping, that can induce crystallization of the material.

### SUMMARY

The present disclosure discloses the use of magnesium or magnesium based alloy, which is loaded with catalytic areas and/or surface treated with catalyst, which reduces the hydrogen gas evolution/release inside a person.

To successfully introduce magnesium and magnesium alloys into a clinical setting it is vital to reduce the total hydrogen released from the implant, not only its hydrogen release rate. The present disclosure solves or at least mitigates the problems described above, by providing implant materials having improved degradation properties, manifested by reduced hydrogen gas emission, compared to conventional magnesium based materials.

The present disclosure provides a material technology that may be used for a great variety of medical applications, such as coronary stents, clips and sutures, vascular closure devices and temporary attachment of pacemaker/neurostimulation means and orthopedic implants.

### BRIEF DESCRIPTION OF THE DRAWINGS

The accompanying drawings illustrate various embodiments of the present disclosure and are a part of the specification. The illustrated embodiments are merely examples of the present disclosure and do not limit the scope of the invention.

FIG. 1 is a schematic illustration of the catalytic conversion of hydrogen on degrading magnesium or magnesium alloy; and

FIG. 2 is a schematic illustration of a composite material according to one embodiment of the present invention.

### DETAILED DESCRIPTION

The present disclosure discloses the use of magnesium or magnesium based alloy, which is loaded with catalytic areas and/or surface treated with catalyst, which reduces the hydrogen gas evolution/release through one or several of the following mechanisms: reduction of the exchange current rate for hydrogen evolution, local and temporary adsorption of hydrogen and/or hydrogen gas, catalytic conversion (oxidation) of hydrogen to water or other compound.

The mechanism for catalytic conversion of hydrogen to reduce total hydrogen release to the body is summarized as follows:

1^{st} step: Hydrogen gas is produced by the degradation of Mg or Mg alloy.
2^{nd} step: Hydrogen is brought into contact with the catalytic site. The contact can be facilitated by one or more different processes, such as (but not limited to) adsorption and chemisorption. Hydrogen can be initially adsorbed by the catalytic site, or initially adsorbed by a site nearby the catalytic site, and then be brought into contact with the catalytic site following desorption.
3^{rd} step: Hydrogen is catalytically oxidized to water (or other compound) on the catalytic site.

This process is illustrated by FIG. 1, where a matrix 1 is depicted, which matrix is made of magnesium or magnesium alloy. At the surface of the matrix 1, the electrochemical reaction as already mentioned above will occur in an aqueous solution (such as is found in tissue and/or blood and/or other fluid inside a patient):

Mg+2H₂O→Mg²⁺+2OH⁻+H₂

In the presence of water molecules 2 (small filled black circles depicting hydrogen atoms and unfilled larger circles depicting oxygen atoms), the magnesium in the matrix 1 will be degraded to magnesium ions 3 (depicted by a large filled black circle). Hydroxide ions 4 and gaseous hydrogen 5 are also formed in the reaction. In the presence of a catalyst 6 (depicted by large unfilled circles) which is dispersed within the matrix 1, the gaseous hydrogen 5 is catalytically converted to water molecules.

According to one aspect of the present disclosure, a composite material is provided which comprises:
a matrix of magnesium or magnesium alloy, and
a catalyst which is dispersed within the matrix,
   wherein the catalyst has the capacity to reduce the amount of hydrogen gas released from the matrix when the matrix is being degraded.

The term “within the matrix” as used herein includes the situation where the catalyst is located on the surface of the matrix, the situation where the catalyst is located below the surface of the matrix, and also the situation where the catalyst is located both on the surface and also below the surface of the matrix. In one embodiment of the present disclosure a catalyst system is incorporated in the metal bulk (magnesium or magnesium alloy) and/or on the surface of the metal bulk to promote hydrogen gas oxidation and potential following reactions of the reactant, wherein the reaction catalyst comprises platinum (Pt) or any one of the platinum group metals ruthenium (Ru), rhodium (Rh), palladium (Pd), osmium (Os), and iridium (Ir) or mixtures thereof. The catalyst, or mixes of catalysts, can be supported on another material, such as carbon black. Hereby, the hydrogen gas is catalytically converted to water or other compound, such as hydrogen ion or hydroxyl ion.

In another embodiment, the catalyst comprises metal oxides and/or carbides, such as vanadium (V) or nickel (Ni) based oxides, W based carbides or mixtures thereof.

In yet another embodiment, the catalyst comprises a compound that has high affinity for hydrogen adsorption and slow reaction kinetics for hydrogen evolution, such as niobium (Nb), molybdenum (Mo), tantalum (Ta) or tungsten (W) or mixtures thereof. These materials will serve as local and temporary storage sites for hydrogen. The hydrogen is thereafter desorbed from the adsorption site and catalytically converted at the catalytic site, which can be the same surface as the adsorption site or located elsewhere on the material. The hydrogen oxidation reaction is commonly catalysed by Platinum (Pt) or other platinum group metals (Pt, Ru, Pd, Ir and Rh) or mixes thereof. The catalyst can be supported on e.g. carbon black. Also Pt can be alloyed or mixed with other elements (including, but not limited to, e.g. Sn, Mo, Ni, Fe, Ag, W, Os, Co, Mn, and their oxides and carbides) to improve the catalytic properties, through various effects, such as bifunctional effects, ligand (electronic) effects and, ensemble (morphological) effects. Additional materials and techniques are described in Chapter 3.3 in, PEM Fuel Cell Electrocatalysts and Catalyst Layers: Fundamentals and Applications, (J. Zhang, author), 2008 Springer Verlag London Limited, whose entire contents are incorporated herein by reference for the materials and techniques described therein.

A composite material according to the present disclosure is illustrated in FIG. 2, depicting a matrix 1 made of magnesium or magnesium alloy, and a catalyst 6 dispersed throughout the matrix 1. The catalyst 6 comprises a catalytic compound or a mixture of different catalytic compounds, as described above, which may be dispersed within the matrix 1 in varying amounts. The catalyst 6 may be uniformly or non-uniformly distributed within the matrix 1. The amounts and distribution patterns of the catalyst are chosen such that there is always an amount of active catalyst present on the surface of the composite material.

The catalysts can be incorporated and dispersed into the material by several processes, such as mixing of catalytic powders into Mg (or Mg-alloy) powder followed by sintering into a material, or mixing of salt of the catalyst into Mg (or Mg-alloy) powder followed by sintering, or dispersion of solutions (aqueous or non-aqueous) containing the catalyst into the Mg (or Mg-alloy) powder followed by sintering. Alternatively, the catalysts may be incorporated into the Mg (or Mg-alloy) melt and if it is not soluble in the melt it will be dispersed into the melt by mixing and/or convection and maintain its structure upon cooling of the melt.

According to another aspect of the present disclosure, a medical implant is provided which comprises a composite material as disclosed above.

Such a medical implant makes it possible to achieve reduced hydrogen evolution while maintaining desirable mechanical and corrosion characteristics related to magnesium based materials. In addition, incorporation of catalysts into the magnesium based material allows for a means to control and tailor the degradation rate as different trace compounds affect the degradation time of magnesium. For example, trace levels of nickel, which is a powerful catalyst for hydrogen oxidation, at the same time greatly accelerates the degradation rate of magnesium.

Yet another aspect of the present disclosure relates to a method for incorporating a catalyst in the metal or metal alloy, including coating the surface of the metal or metal alloy with a catalyst, comprising sintering a mixture of the bulk or matrix material (Mg-based) and one or several catalysts. The sintering can be performed on powder mixtures by conventional sintering or by other forms of sintering, like spark plasma sintering, liquid phase sintering (addition of a material with a lower melting point than the matrix to be sintered, and electric current assisted sintering.

The catalyst (in powder form) can be directly mixed into the bulk powder material by mechanical means. An alternative method to deposit the catalyst is to first dissolve the catalyst in liquid (e.g. salt of Pt (e.g. H₂PtCl₆.6H₂O, or H₃Pt(SO₃)₂OH) in water, diluted acid or organic solvent), and then to deposit the catalyst, with or without the use of electrochemical assistance, onto the powder bulk material. The excess solvent is removed by evaporation, optionally assisted by heating. This is suitable when the catalyst material has a higher standard reduction potential (i.e. is more noble) than the (Mg) bulk material, as in the case of for example Pt, Pd, Ru, etc. (Bitter et al., Catalysis Today, 29 (1996) 349-353; Thompson et al., Journal of Electroanalytical Chemistry 515 (2001) 61-70).

Thus, the catalyst will be dispersed throughout the entire magnesium-based matrix. Consequently, there will always be active catalyst present on the surface, ready to take care of the hydrogen gas formed as the degradation of the magnesium-based material progresses. The catalyst will only become active when it appears on the surface of the material.

The implantable components can be directly produced by sintering into a desired shape or sintered into a basic shape and then reshaping and modifying into final shape, by for example grinding, rolling, or other metal shaping/processing method.

Specific applications of the present disclosure include the use of degradable coronary (or other blood contacting) stents in diabetic patients. In general, diabetic patients have a high risk of rapid restenosis (i.e. re-narrowing of blood vessels) and degradable stent technologies (see e.g. US2005010279A1) have been proposed to improve the outcome for these patients. The entire contents of this publication is incorporated herein by reference for the medical devices and related processes described therein.

Another application of the present disclosure is extravascular closure devices in the form of metal clips for rapid and secure closure. The metal clip technique is advantageous as it has high mechanical stability, and is degradable and radio-opaque. The metal clip can, for example, be used to seal the hole in the vessel following a procedure requiring an arterial puncture. If the patient requires another arterial puncture before the clip is degraded it can easily be visualized by X-ray prior to the procedure, and the vessel can be punctured in another location to avoid the remaining clip being pushed into the vessel. X-ray visualisation is not possible with today's degradable closure devices as they are not X-ray visible.

Another application of the present disclosure is vascular closure devices in the form of metal components for rapid and secure closure. The vascular closure device can comprise two degradable components (one or both made from degradable metal), one of which is located inside and one outside the blood vessel. The two components are then joined together (by for example a suture) to obtain haemostasis by mechanically blocking the hole in the vessel. Existing vascular devices use degradable polymers but degradable metal components are advantageous as they have more suitable mechanical properties, and are radio-opaque. Examples of such closure devices are described in detail in U.S. Pat. Nos. 6,090,130; 6,508,828; 7,931,670; 7,250,057; 7,713,283; and 7,988,706. The entire contents of these patents are incorporated herein by reference for the devices, components, materials, and processes described therein.

The invention also includes use of a composite material as described herein for reducing the release of hydrogen gas from the matrix when the matrix is being degraded in an aqueous environment at a pH below 11.5.

The present disclosure is not limited to the above-described preferred embodiments. Various alternatives, modifications and equivalents may be used. Therefore, the above embodiments should not be taken as limiting the scope of the invention, which is defined by reference to the appended claims.

1. A composite material comprising:
a matrix of magnesium or magnesium alloy, and
a catalyst which is dispersed within the matrix,
wherein the catalyst has the capacity to reduce an amount of hydrogen gas released from the matrix when the matrix is being degraded.

2. A composite material according to claim 1, wherein the matrix is adapted to being degraded in an aqueous environment at a pH below 11.5.

3. A composite material according to claim 1, wherein the catalyst is selected from the group consisting of platinum (Pt), ruthenium (Ru), rhodium (Rh), palladium (Pd), osmium (Os), iridium (Ir), niobium (Nb), molybdenum (Mo), tantalum (Ta), tungsten (W), metal oxide, vanadium (V) based oxide, nickel (Ni) based oxide, or mixtures thereof.

4. A composite material according to claim 1, wherein 0.001-1% by weight of the composite material consists of the catalyst.

5. A composite material according to claim 1, wherein the catalyst is dispersed throughout the entire matrix of magnesium or magnesium alloy.

6. A composite material according to claim 1, wherein the catalyst is dispersed throughout the matrix such that there is always active catalyst present on a surface of the matrix.

7. A medical implant comprising a composite material according to claim 1.

8. The medical implant according to claim 7, wherein the medical implant is selected from the group consisting of a coronary stent, a suture, a vascular closure device, a device for temporary attachment of a pacemaker or a neurostimulator, an orthopedic implant, an extravascular closure device, and a clip.

9. A method for preparing a composite material according to claim 1, comprising dispersing a catalyst into the matrix by sintering a mixture of the matrix and the catalyst.

10. A method for producing a medical implant according to claim 7, comprising sintering the medical implant into a desired shape directly, or sintering the medical implant into a basic shape, followed by reshaping it into its final shape.

11. A method for producing a medical implant according to claim 10, wherein the reshaping includes at least one of grinding and rolling.

12. A method for reducing release of hydrogen gas inside a patient, comprising:
inserting a medical implant comprising the composite material according to claim 1 inside a patient.
